(19) United States
(12) Patent Application Publication
(10) Pub. No.: US 2009/0324555 A1
(43) Pub. Date: Dec. 31, 2009

(54) Neural Stem Cells

(75) Inventors: Michael Thie, Bonn (DE); Özer Degistirici, Bonn (DE)

Correspondence Address: JOYCE VON NATZMER;PEQUIGNOT + MYERS LLC, 200 Madison Avenue, Suite 1901, New York, NY, 10016, US

(73) Assignee: STIFTUNG CAESAR, Bonn (DE)

(21) Appl. No.: 11/909,188

(22) PCT Filed: Mar. 23, 2006
(86) PCT No.: PCT/EP2006/002694

§ 371 Date: Sep. 4, 2009

(30) Foreign Application Priority Data
Mar. 23, 2005 (EP) 05102356.2

(51) Int. Cl. A61K 35/12; C12N 5/00; A61P 25/00

(52) U.S. Cl. 424/93.7; 435/377; 435/325

(57) ABSTRACT

Subject of the invention is a method for generating neural stem cells in vitro, wherein dental progenitor cells are isolated from soft tissue of tooth or wisdom tooth and cultivated until they form primary spheres which are then dissociated into single cells. These single cells are cultivated until they form spheroids and the spheroid-forming cells are separated to obtain neural stem cells.

### FIELD OF THE INVENTION

Subject of the invention is an in vitro method for generating neural stem cells from dental progenitor cells isolated from soft tissue of tooth or wisdom tooth.

### BACKGROUND OF THE INVENTION

Neurodegenerative diseases are characterized by the loss of specific subsets of neurons, and whilst drug therapies exist for some of these disorders, none of them are curative. Neural tissue has a limited capacity for repair after injury, and adult neurogenesis is limited to selected regions of the brain (Gage, 2000; Magavi, 2000; Rakic, 2000 and Temple and Alvarez-Buylla, 1999).

Neural cells can be generated from embryonic stem cells (ESCs) and neural stem cells (NSCs) from embryonic tissue (Bain et al., 1995; Bruestle et al., 1999; Lindvall et al., 1990; McKay, 1997) or from fetal tissues, the most successful being the transplantation of human fetal tissue into Parkinson's patients (Freed et al., 2001).

The use of ESCs and NSCs which are derived from embryonic and fetal tissues is limited by various ethical and logistical constraints, and thus tissues from adults may be an alternative source of stem cells and progenitor cells as shown in the case of bone marrow (Brazelton et al., 2000; Mezey et al., 2003; Sanchez-Ramos et al., 2002; Jiang et al., 2002) and from developing (Vescovi et al., 1999; Svendsen et al. 1997) or adult brain (Studer et al., 1998; Wu et al., 2002; Daadi and Weiss, 1999; Vicario-Abejon et al., 2000). Further sources already available include bone marrow stromal cells (Prockop et al., 2000; Hofstetter et al., 2002), stem cells from dermis (Toma et al., 2001) and neural crest stem cells from the gut and sciatic nerve (Bixby et al., 2002; Kruger et al., 2002).

### PIOR ART

It has recently been shown that exfoliated human deciduous teeth and dental pulp contains a population of multipotent stem cells with the capacity to differentiate into several different cell lineages, including glial and nerve cells (Miura et al., 2003; Gronthos et al., 2002).

In WO 03/066840 it is disclosed that adherent growing pluripotent embryonic-like stem cells derived from dental follicle of teeth have been isolated. The inventors found that the pluripotent stem cells have the potential for extended renewal of teeth, periodontium and related tissues such as bones. The cells are cultured as biological membranes or scaffolds, which are necessary to support differentiation into mesothelic/endothelic cells, into blood vessels, into ectodermal tissue or into neural tissue of teeth (periodontium).

So far, the work with human embryonic stem cells in cell replacement therapies has been mostly hampered by the low cell numbers isolated from human fetal tissues and further by ethical and technical problems to produce these cells (Bjorklund et al., 2002).

### SUMMARY OF THE INVENTION

The problem underlying the invention is thus to provide an alternative method for obtaining neural stem cells. The stem cells should be easily available without ethical or technical constraints, be obtainable in a simple process in high yield, and be fully capable of differentiation to neuronal cells.

Surprisingly, the problem of the invention is solved by a method comprising the steps of
cultivating said dental progenitor cells until they form primary spheres,
dissociating said primary spheres into single cells,
cultivating said single cells until they form spheroids, and
separating the spheroid-forming cells to obtain neural stem cells.

The invention comprises the isolation and expansion of multi-potent stem cells from the ectomesenchymal soft tissue of the third molar (wisdom tooth), which forms spheroids and progenitor cells with neuronal differentiation capacity.

According to the present invention, it is possible to directly generate neural stem cells from dental follicle without “membrane formation”. “Free cells” in the context of the invention means that the NSC for isolation and cultivation are not embedded in a matrix, like a membrane or tissue. It is not necessary to use such a differentiation process to produce the neural stem cells. The invention is not related to the generation of periodontal neuronal cells as disclosed in WO 03/066840. The free stem cells of the invention may be single NSCs or homogenous cell aggregates like spheroids and neurospheres.

The procedure of the invention is suitable to generate a population of neural stem cells (NSCs), which are ectomesenchymal-derived dental follicle cells. According to the invention, cell aggregates (speroids, neurospheres) are formed from the dental follicle derived cells by neurogenic stimulation and acquire clear neuronal morphology and protein expression profile in vitro. This indicates the presence of a cell population in the dental follicle of the third molar with neuronal differentiation capacity that might provide benefits when implanted into central and peripheral nerve system.

The invention provides a method for obtaining neural stem cells, wherein stem cells are obtained from tissue from the dental follicle of tooth or wisdom tooth and differentiated to the neural stem cells. Such stem cells from tissue of the dental follicle are known from WO 03/066840. They can be isolated by the methods disclosed in WO 03/066840, especially the example on pages 20/21, which are incorporated herein by reference.

However, WO 03/066840 does not disclose the generation of neural stem cells. It only teaches that the dental follicle stem cells could be used for the creation of periodontal cell lines. In contrary, the free NSCs obtainable according to the invention are not limited to applications associated with the periodontium. This finding is very surprising, because in adult tissue like wisdom tooth there seems to be no need for stem cells capable of differentiation into cells which are not periodontal.

According to the invention, free neural stem cells are available which do not have to be generated in a tissue, biological membrane or scaffold. It is not necessary to add such tissues or membranes to promote differentiation.

Neural stem cells generated from wisdom teeth tissue are easily available and can be expanded in two strategies, i.e. from single spheres to a suspension culture of several spheres (i) or from single spheres to monolayer cultures and back to suspension cultures (ii). By this, a large number of cells can be generated to use for cell replacement strategies in treatment of neurodegenerative diseases. Furthermore, the neural induction medium used here allows growth and differentiation of neural cell types derived from spheroid-forming NSCs.

The availability of NSCs derived from cells from the soft tissue of wisdom tooth (dental follicle) and the ability to differentiate into neurons, astrocytes or cholinergic neurons makes these cells ideal candidates for cell replacement therapies in neurodegenerative disorders, like Parkinson's disease or amyotrophic lateral sclerosis.

According to the invention, the primary spheres and single cells, respectively, are cultured under sphere-forming conditions that are preferably established by a medium comprising both bFGF and EGF. This medium may further comprise B27 (1:50) or ITS+Remix (1:50) and/or neurobasal medium or DMEM High Glucose.

The neural stem cells according to the invention are differentiated to neural cells by incubation in differentiation-medium, preferably on coated flasks or cover slips which are preferably coated with fibronectin or poly-D-lysine and laminin.

In a preferred embodiment of the method according to the invention, the dental progenitor cells are isolated from ectomesenchymal soft tissue of tooth or wisdom tooth, for example, dental follicle and/or apical soft tissue.

The invention further concerns a neural stem cell generated by the method according to the invention and a differentiated neural cell produced by the method according to the invention. The invention also comprises a cell culture or structure comprising at least one neural stem cell according to the invention and/or one differentiated neural cell according to the invention as well as a pharmaceutical composition comprising at least one neural stem cell according to the invention and/or one differentiated neural cell according to the invention.

According to the invention, the dental progenitor cells obtained from soft tissue, preferably ectomesenchymal soft tissue, of tooth or wisdom tooth may be used for the production of neural stem cells in vitro.

At least one neural stem cell according to the invention and/or at least one differentiated neural cell according to the invention, or the pharmaceutical composition according to the invention, may be used for the treatment of neurodegenerative diseases, for example, Alzheimer's disease, Parkinson's disease, prion diseases, Creutzfeldt-Jakob, Huntington's disease, multiple sclerosis, frontotemporal dementia (Pick's Disease) or amyotrophic lateral sclerosis (ALS or Lou Gehrig's Disease).

The invention is exemplary described below in detail with reference to the drawings.

### BRIEF DESCRIPTION OF THE DRAWINGS

In the figures:

FIG. 1 shows initially formed spheres in primary culture a, b;

FIG. 2 shows spheres proliferated in neurobasal medium containing bFGF, EGF, B27;

FIG. 3 shows a sphere with cilia in perimetry (400×);

FIG. 4 shows a sphere formed in DMEM-high glucose containing ITS+premix, bFGF, EGF (a), and a sphere formed in neurobasal medium containing B27, EGF, bFGF (b);

FIG. 5 shows spheres cultivated in neurobasal medium (a), mechanically dissociated and cultivated in FCS containing medium grown in monolayer (b), a monolayer trypsinized and cultivated in neurobasal medium (c), and in ITS+premix medium (d); The cells can be grown again in spheres;

FIG. 6 shows spheres formed initially in primary culture, positively stained with p75 antibody;

FIG. 7 shows NSCs treated with differentiation medium;

FIG. 8 shows neurofilament stained cells;

FIG. 9 shows GFAP stained cells;

FIG. 10 shows GABA stained cells;

FIG. 11 shows choline acetyl transferase (CAT) stained cells;

FIG. 12 shows an intensity plot representation of RNA expressed in neuro-genic stimulated tooth derived neural stem cells in a patient (P1);

FIG. 13 shows an intensity plot representation of RNA expressed in neuro-genic stimulated tooth derived neural stem cells in another patient (P2);

FIG. 14 shows an intensity plot representation of RNA expressed in un-stimulated tooth derived neural stem cells in a patient (P2), control.

### VARIOUS AND PREFERRED EMBODIMENTS OF THE INVENTION

Cells were enzymatically isolated from dissected soft tissue of wisdom teeth (dental follicle or apical soft tissue) by collagenase/Dispase treatment.

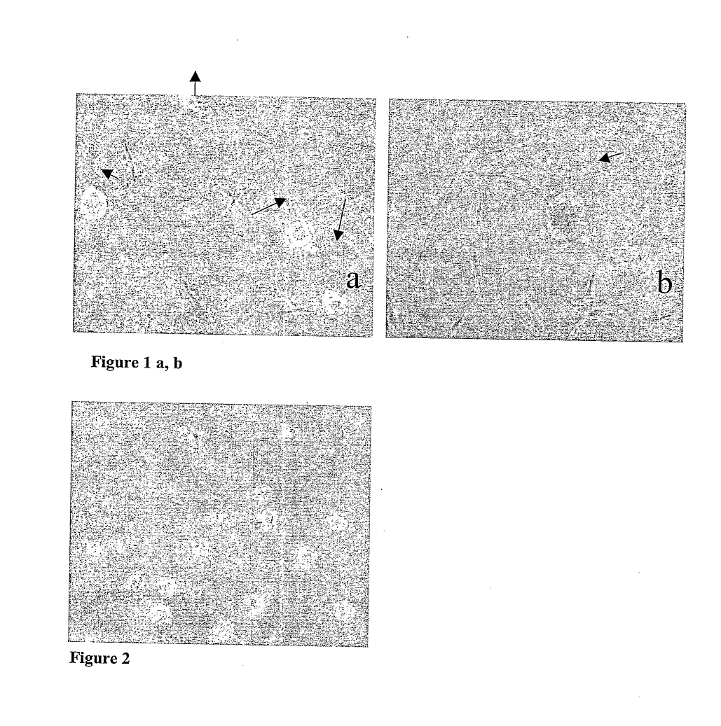

The ectomesenchymal cells were cultivated in FCS containing medium for 8-12 days. Some of the cells adhered to the plastic culture flask while some died in suspension. However, some of the cells formed spheres (FIG. 1).

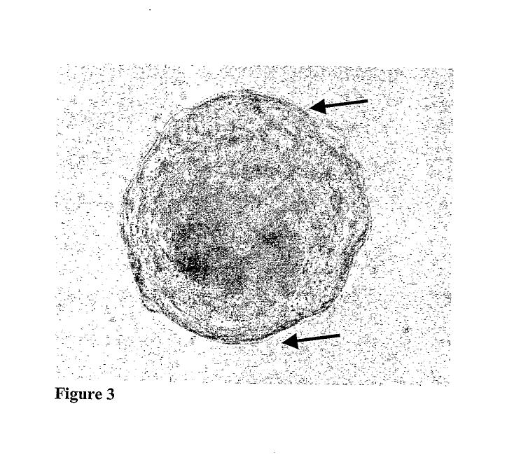

The floating spheres were transferred to new culture flasks after initial culturing in bFGF (40 ng/ml) and EGF (20 ng/ml), B27 (1:50) and neurobasal medium (Invitrogen) containing medium or bFGF (50 ng/ml), EGF (25 ng/ml), ITS+Premix (1:50) and DMEM High glucose containing medium. Cells in spheres proliferated thereby forming large spheres which were successfully passaged and expanded (FIG. 2). The spheres seemed bright when viewed under a phase contrast microscope and showed cytoplasmic protrusions (cilia) at their surface (FIG. 3).

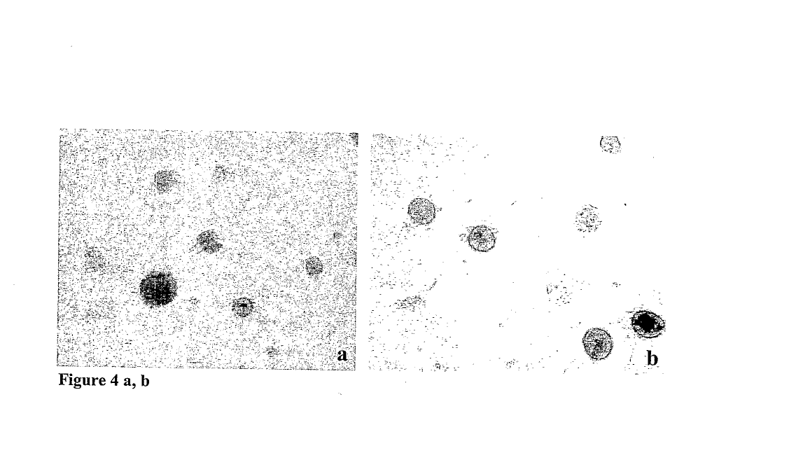

The primary spheres were mechanically dissociated into single cell suspensions and cultured again under sphere-forming conditions (ITS+Premix, bFGF, EGF, DMEM HG or bFGF, EGF, B27 and neurobasal medium; FIGS. 4a, b).

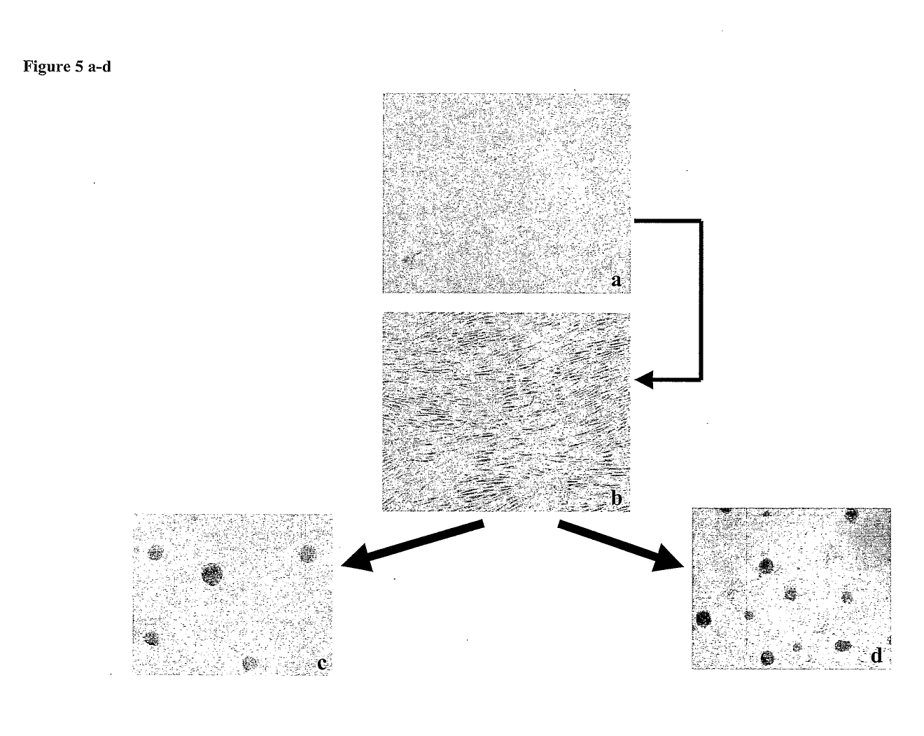

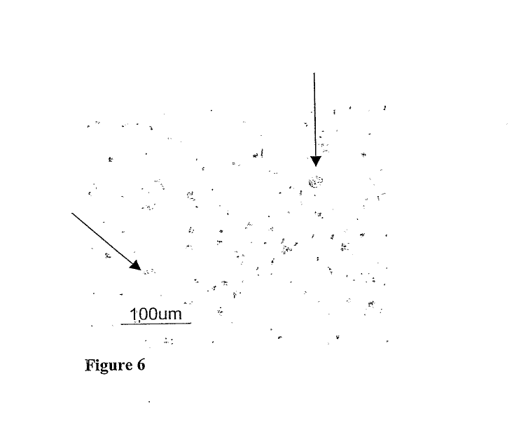

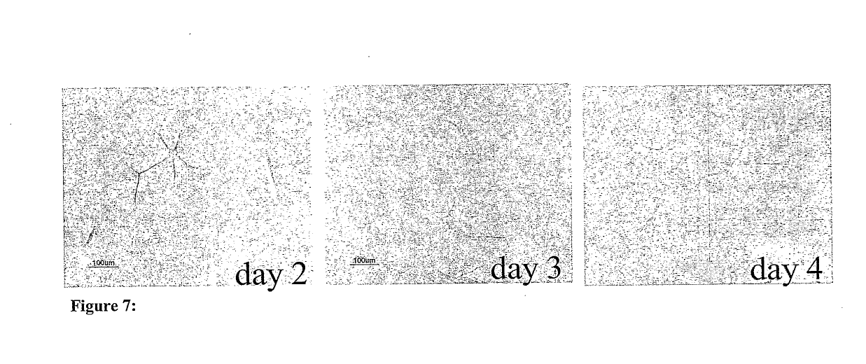

After each passage with both media the number of cells adhering to the plastic flask decreased while the number of cells forming spheroids increased (FIG. 5a). When the spheres grew in DMEM basic medium supplemented with serum, undifferentiated stem cells retained a flat polygonal fibroblast-like morphology (FIG. 5b), in serum-free culture conditions cells formed again spheres (FIGS. 5c, d). Immunocytochemistry with undifferentiated NSCs spheroids revealed that cells were stained for p75, a marker for neural growth factor receptor (FIG. 6).

The potency of ex vivo expanded wisdom teeth-derived neural stem cells to generate neural cells was analyzed. The stem cells were seeded on fibronectin-coated glass cover slips. Cells were incubated in differentiation-medium containing DMEM, 15% heat inactivated FCS, penicillin/streptomycin/glutamine, 50 ng/ml β-NGF, 20 ng/ml FGF-b, 1 mM dibutyryl cAMP, 0.5 mM 3-isobutyl-1-methylxanthine, 10 μM all trans-retinoic acid. Differentiation-medium was changed every second day. After each day, cells showed neuronal morphology (FIG. 7).

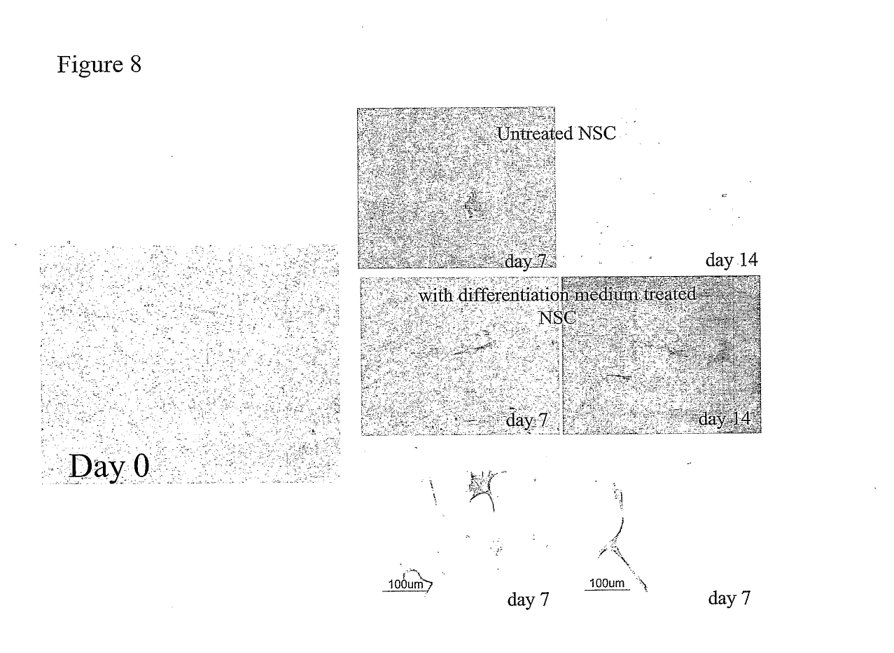

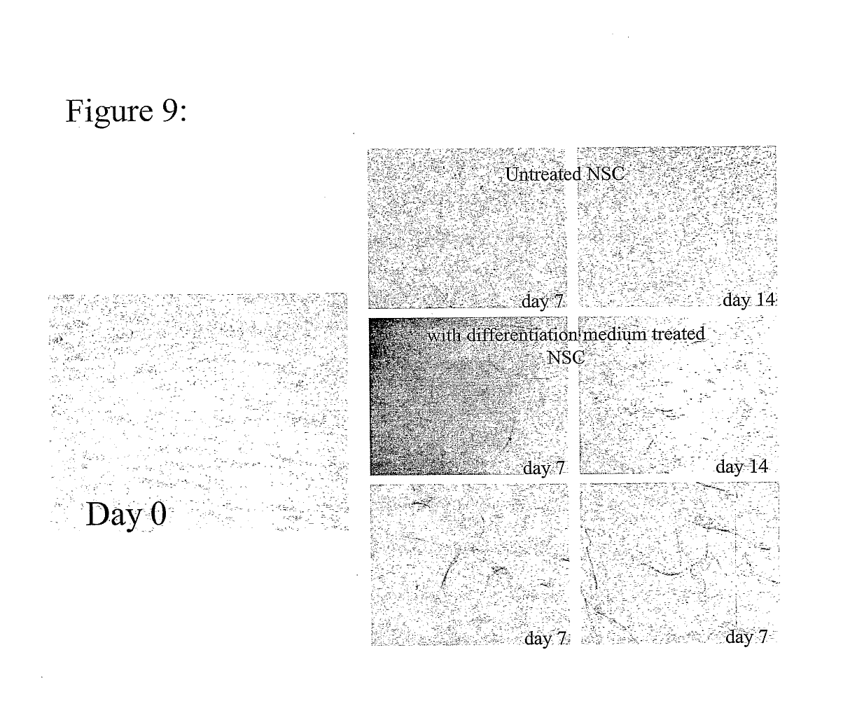

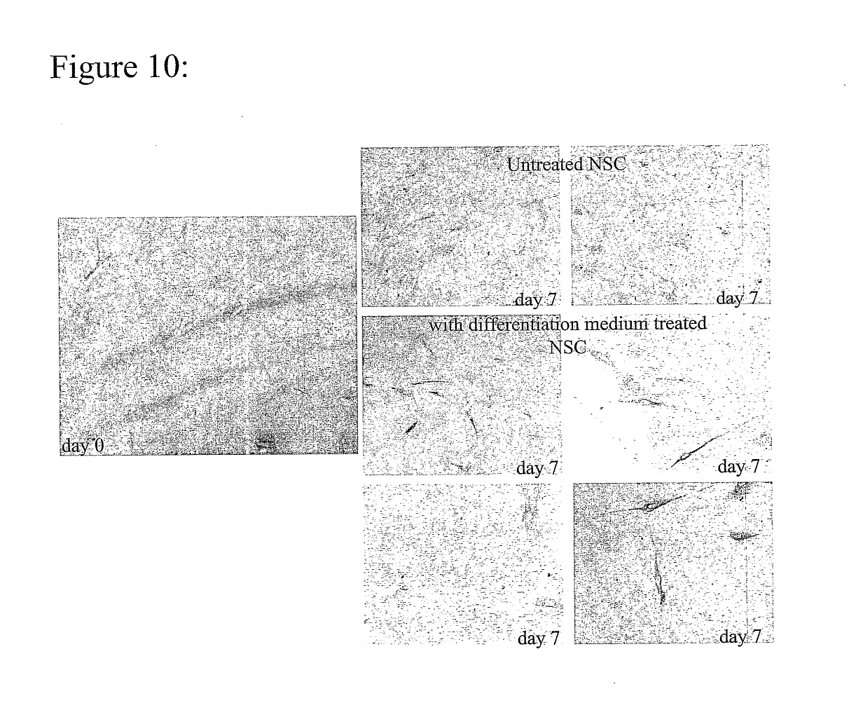

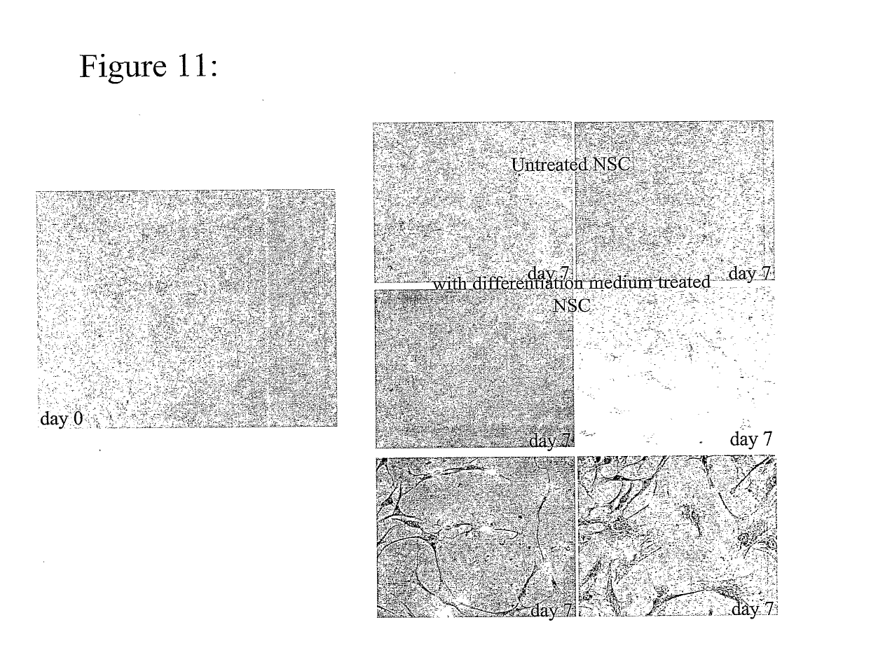

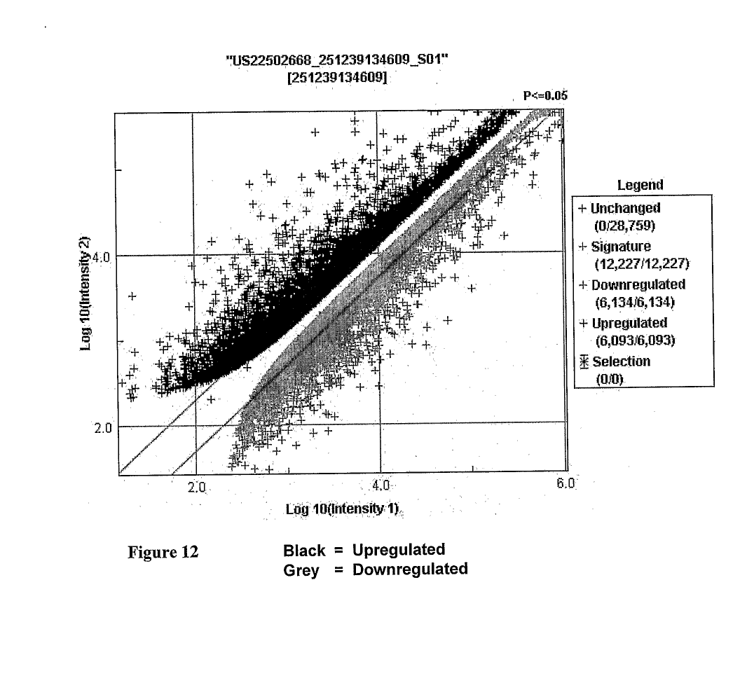

After one and two weeks, respectively, of differentiation-medium incubation, a subpopulation of neural stem cells was stained for neurofilament, a marker of postmitotic neurons (FIG. 8). Neural stem cells can be differentiated after one and two weeks into the glial lineage, expressing GFAP, a protein of the astrocytic cytoskeleton (FIG. 9). A low percentage of NSCs showed staining for the inhibitory neurotransmitter GABA in basic growth medium after seven days of culture, indicating spontaneous differentiation (FIGS. 10b, c). At day 0, the cells were negative for GABA (FIG. 10a). The staining for the inhibitory neurotransmitter GABA significantly increased by incubation in differentiation-medium (FIGS. 10d, e, f, g). After seven days of stimulation of NSCs with differentiation-medium, immunoreactivity for choline acetyltransferase (CAT) was found indicating the development of a cholinergic neuronal subtype (FIG. 11).

In order to further analyse the fundamental molecular processes involved in neurogenic differentiation of teeth-derived neural stem cells, a microarray analysis of the critical stages during in vitro stimulation of cells was accomplished. Cells from two volunteers (patient 1: age: 14, gender: w; patient 2: age: 12, gender: w) were isolated from apical, ectomesenchymal soft tissue (apical pad) of tooth and then cultured as described above. Primary spheres were dissociated into single cell suspensions and cultivated in poly-D-lysine and laminin coated plastic flasks for 14 days with neurogenic growth medium (DMEM, 15% heat inactivated FCS, penicilin/streptomycin/glutamin, 50 ng/ml β-NGF, 20 ng/ml FGF-b, 1 mM dibutyryl cAMP, 0.5 mM 3-isobutyl-1-methylxanthine, 10 μM all trans retinoic acid) and with medium omitting growth factors, respectively. RNA isolation was performed on each sample using the RNeasy Mini kit (Qiagen). The mRNA of each sample was isolated and amplified according to the manufacturer's protocol (Qiagen). Each sample was then submitted to an Agilent core facility (caesar), where hybridization of the RNA to the chip probes and fluidics were completed using the standard Agilent gene chip analysis protocol. For analysis a 39.000 oligonucleotide platform was used (Agilent). Raw data were compiled with Agilent software and analysis was achieved using Rosetta software (Agilent).

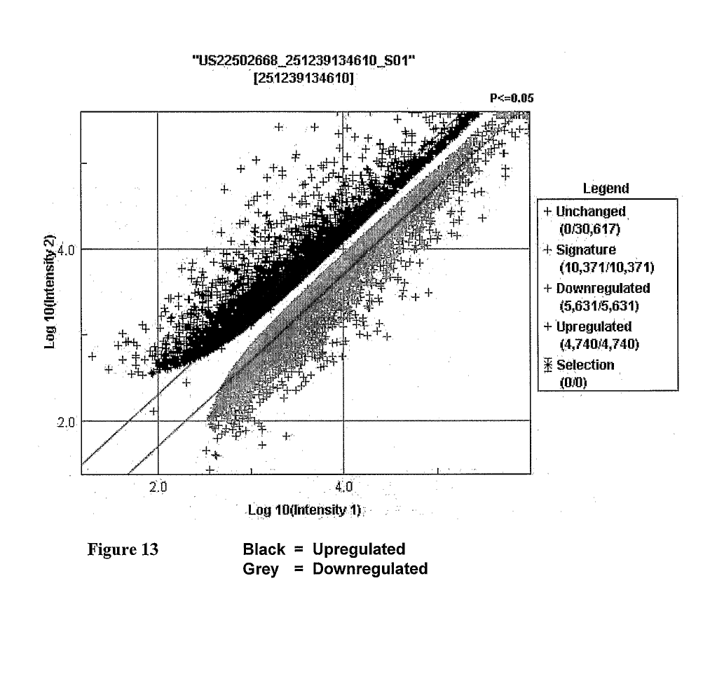

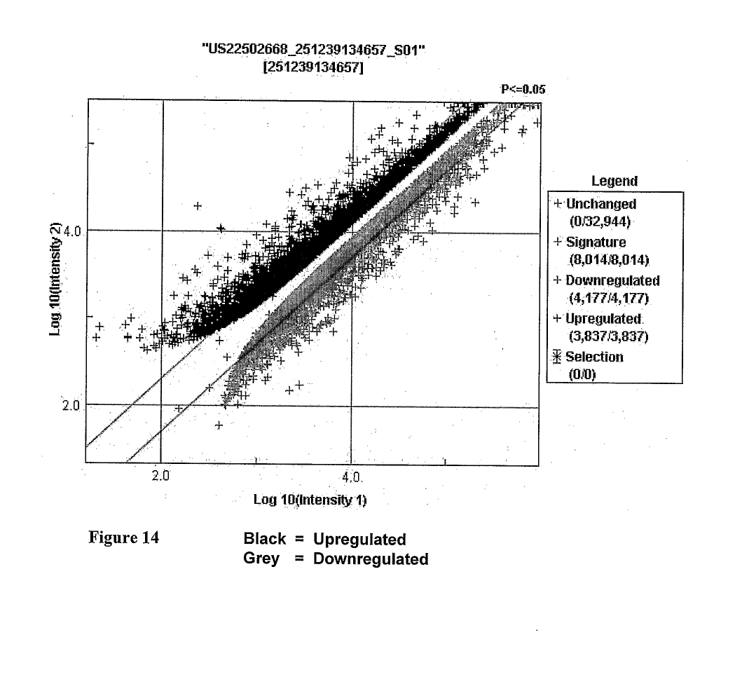

A readout of gene chips from patient 1 (with stimulation) shows 6.093 genes upregulated, 6.134 genes downregulated and 28.759 genes unchanged. Patient 2 (with stimulation) shows 4.740 genes upregulated, 5.631 downregulated while 30.671 genes remained unchanged. The matched control (without stimulation) shows 3.837 genes upregulated, 4177 downregulated while 32.944 genes were unchanged. With regard to patient 2, 2.357 genes were regulated in teeth-derived neural stem cells when cultured in neurogenic growth medium for 14 days. See graphic analysis of expression changes in FIG. 12 (patient 1) and FIGS. 13 and 14 (patient 2). Only genes with p values less than 0.05 were used for individual gene analysis to ensure quality of data.

Next, specifically regulated genes increasing from 1.4-fold to 100-fold (patient 2) and from 3-fold to 100-fold (patient 1) were listed. In particular, transcripts that showed threefold or higher changes in expression included neuronal or proneuronal markers (neurotrophic tyrosine kinase-receptor, neurokinin-1, latexin, neuromedin-U receptor-1, tubulin, beta polypeptide paralog, neurofilament 3, myelin expression factor 2, leukemia inhibitory factor (cholinergic differentiation factor (LIF)) and mRNAs encoding WNT proteins (WNT2, WIF1, WNT5A), which are key regulators of neural stem cell behaviour in embryonic development and with other genes, such as promotor nerve precursor differentiation (Spondin-1), Amphiregulin, a mitogen for adult neural stem cells, other genes such as those involved with the development and differentiation of mature neural cells (IGF1, BMP2, HES1, retinoic acid receptors, TGFβs), also showing upregulation of fivefold or greater. Large numbers of in central nerve system (CNS) expressed transcripts were also upregulated at high levels (i.e., 3- to 36.0-fold change), notably those encoding the proteins brain expressed X-linked proteins (BEX1, BEX2), GABA(A) receptor-associated protein like, GABA-A receptor-associated protein, Selenoprotein P, neurite growth-promoting factor 1, gamma-aminobutyric acid (GABA) A receptor, epsilon (GABRE), solute carrier family 1 (glial high affinity glutamate transporter), member 3 (SLC1A3), serotonin, Dopamine receptor D4, brain-derived neurotrophic factor (BDNF), cerebellar degeneration-related protein 1 (see Table 1 where up-regulations of genes, patient 1+2, are matched against unstimulated cells, patient 2). Only stimulated cells show increase in expression of neural/glial markers while unstimulated cells did not.

These results demonstrate that the teeth-derived neural stem cells according to the invention may differentiate to cells with characteristics not only of neuronal and glial cells but also of CNS cells such as dopaminergic, serotonergic, and GABA-ergic neurons. Thus, teeth-derived neural stem cells might be an excellent source of cells for treatment of neurodegenerative disorders.

TABLE 1
Comparison of absolute gene expression levels in neurogenic stimulated
neural stem cells from tooth (Patient 1 and 2) and 14 days cultivated
and untreated neural stem cells from tooth (Patient 2)

| | | Fold | Fold | Fold |
|---|---|---|---|---|
| | | Change | Change | Change |
| | | Patient-1 | Patient-2 | Patient-2 |
| | Accession | (neural | (neural | control |
| Gene Description | Number | stimulated) | stimulated) | (unstimulated) |
| amphiregulin (schwannoma-derived | NM_001657 | 100 | 100 | 2.9 |
| growth factor) (AREG) | | | | |
| spondin 1, extracellular matrix | NM_006108 | 100 | 92 | 1.2 |
| protein (SPON1) | | | | |
| latexin (LXN) | NM_020169 | 100 | 100 | 1.2 |
| retinoic acid receptor responder | NM_002888 | 47 | 17 | 2.9 |
| (tazarotene induced) 1 (RARRES1) | | | | |
| insulin-like growth factor binding | NM_000599 | 43 | 28 | 1 |
| protein 5 (IGFBP5) | | | | |
| brain expressed X-linked 2 (BEX2) | NM_032621 | 36 | 27 | 2.1 |
| brain expressed, X-linked 1 (BEX1) | NM_018476 | 35 | 33 | 1.2 |
| wingless-type MMTV integration | NM_003391 | 33 | 25 | −1.2 |
| site family member 2 (WNT2), | | | | |
| selenoprotein P, plasma, 1 | NM_005410 | 32 | 15 | 1.005 |
| (SEPP1) | | | | |
| WNT inhibitory factor 1 (WIF1) | NM_007191 | 29 | 19 | 1.7 |
| chemokine orphan receptor 1 | NM_020311 | 28 | 92 | 6.2 |
| (CMKOR1) | | | | |
| retinoic acid receptor, beta (RARB) | NM_000965 | 27 | 19 | 1.8 |
| chemokine (C-X-C motif) ligand 1 | NM_001511 | 26 | 14 | 1.3 |
| (melanoma growth stimulating | | | | |
| activity, alpha) (CXCL1), | | | | |
| retinol dehydrogenase 10 (all-trans) | NM_172037 | 25 | 14 | −1.06 |
| (RDH10) | | | | |
| solute carrier family 1 (glial high | NM_004172 | 21 | 15 | −1.8 |
| affinity glutamate transporter), | | | | |
| member 3 (SLC1A3) | | | | |
| GABA(A) receptor-associated | NM_031412 | 18 | 12 | 1.6 |
| protein like 1 (GABARAPL1) | | | | |
| synuclein, alpha interacting protein | NM_005460 | 16 | 9 | 1.4 |
| (synphilin) (SNCAIP), | | | | |
| Tissue factor pathway inhibitor 2 | AK129833 | 16 | 9 | 1.06 |
| precursor (TFPI-2) (Placental | | | | |
| protein 5) (PP5) | | | | |
| integrin beta 3 | S70348 | 15 | 14 | 2.9 |
| wingless-type MMTV integration | NM_003392 | 14 | 10 | 1.6 |
| site family, member 5A (WNT5A) | | | | |
| Angiopoietin-like 4 (ANGPTL4), | NM_139314 | 12 | 12 | 4.1 |
| spermidine/spermine N1- | NM_002970 | 12 | 9.6 | 3.6 |
| acetyltransferase (SAT) | | | | |
| hairy and enhancer of split 1, | NM_005524 | 12 | 7.5 | 25 |
| (Drosophila) (HES1) | | | | |
| neurotrophic tyrosine kinase, | NM_002529 | 12 | 12.5 | 1.4 |
| receptor, type 1 (NTRK1) | | | | |
| interleukin 8 (IL8) | NM_000584 | 11 | 3.5 | 1.1 |
| tachykinin, precursor 1 (substance | NM_013996 | 11 | 12 | −1.3 |
| K, substance P, neurokinin 1, | | | | |
| neurokinin 2, neuromedin L, | | | | |
| neurokinin alpha, neuropeptide K, | | | | |
| neuropeptide gamma) (TAC1) | | | | |
| pleiotrophin (heparin binding growth | NM_002825 | 10 | 14 | 2.2 |
| factor 8, neurite growth-promoting | | | | |
| factor 1)(PTN), | | | | |
| Voltage-gated calcium channel | THC2054079 | 9 | 6.5 | −1.6 |
| alpha(2)delta-3 subunit | | | | |
| angiopoietin-like 1 (ANGPTL1) | NM_004673 | 9 | 9.2 | 5.3 |
| GABA-A receptor-associated | AF180519 | 8 | 6.7 | 0 |
| protein | | | | |
| bone morphogenetic protein 2 | NM_001200 | 7 | 10 | 2.4 |
| (BMP2) | | | | |
| SRY (sex determining region Y)- | NM_003107 | 7 | 5 | 2.1 |
| box 4 (SOX4) | | | | |
| transforming growth factor, beta 3 | NM_003239 | 7 | 2.5 | 3 |
| (TGFB3) | | | | |
| melanoma associated antigen | NM_152423 | 6 | 17 | 0 |
| (mutated) 1-like 1 (MUM1L1) | | | | |
| neuroblastoma, suppression of | NM_182744 | 6 | 9 | 1 |
| tumorigenicity 1 (NBL1) | | | | |
| 3′,5′-cyclic AMP phosphodiesterase | L12686 | 6 | 6 | 1 |
| cerebellar degeneration-related | NM_004065 | 6 | 5.6 | 4.3 |
| protein 1, 34 kDa (CDR1) | | | | |
| insulin-like growth factor 1 | NM_000618 | 6 | 28 | −1.09 |
| (somatomedin C) (IGF1) | | | | |
| matrix metalloproteinase 10 | NM_002425 | 6 | 11 | 4.3 |
| (stromelysin2) (MMP10) | | | | |
| transforming growth factor, beta 2 | NM_003238 | 6 | 5 | −1.2 |
| (TGFB2) | | | | |
| myeloid leukemia factor 1 (MLF1) | NM_022443 | 6 | 7 | −1.7 |
| gamma-aminobutyric acid (GABA) | NM_021990 | 6 | 5 | −1.2 |
| A receptor, epsilon (GABRE) | | | | |
| matrix metalloproteinase 3 | NM_002422 | 5 | 1.4 | −16.8 |
| (stromelysin 1, progelatinase) | | | | |
| (MMP3) | | | | |
| neuromedin U receptor 1 (NMUR1) | NM_006056 | 5 | 5 | 1.3 |
| platelet derived growth factor D | NM_025208 | 5 | 3 | 3.4 |
| (PDGFD) | | | | |
| platelet-derived growth factor | NM_006207 | 5 | 3 | 2.7 |
| receptor-like (PDGFRL) | | | | |
| interleukin 11 (IL11) | NM_000641 | 5 | 11 | −1.8 |
| meteorin, glial cell differentiation | NM_001004431 | 5 | 4.4 | −1.22 |
| regulator-like (METRNL) | | | | |
| neuropilin 2 (NRP2) | NM_201266 | 5 | 4 | −1.14 |
| Dopamine receptor D4 (Fragment) | THC2173240 | 5 | 2.8 | 1.1 |
| leukemia inhibitory factor | NM_002309 | 3.7 | 3.8 | 2.4 |
| (cholinergic differentiation factor) | | | | |
| (LIF) | | | | |
| tubulin, beta polypeptide paralog | NM_178012 | 3.5 | 2.7 | −1.6 |
| (MGC8685) | | | | |
| brain-derived neurotrophic factor | NM_170735 | 3 | 3 | −1.5 |
| (BDNF) | | | | |
| 5-hydroxytryptamine (serotonin) | NM_019859 | 3.2 | 2.3 | −1.3 |
| receptor 7 | | | | |
| SRY (sex determining region Y)- | NM_007084 | 3 | 1.8 | 1.4 |
| box 21 (SOX21) | | | | |
| snail homolog 1 (Drosophila) | NM_005985 | 3 | 3.3 | 2.6 |
| (SNAI1) | | | | |
| neurofilament 3 (150 kDa medium) | NM_005382 | 3 | 8 | −1.3 |
| (NEF3) | | | | |
| fibroblast growth factor 7 | NM_002009 | 3 | 1.9 | 1.4 |
| (keratinocyte growth factor) (FGF7) | | | | |
| myelin expression factor 2 (MYEF2) | NM_016132 | 3 | 1.7 | −1.3 |
| Purkinje cell protein 4 (PCP4) | NM_006198 | 1.4 | 2.8 | 1.1 |
| nerve growth factor receptor | NM_014380 | 2 | 1.5 | −1.6 |
| (TNFRSF16) associated protein 1 | | | | |
| (NGFRAP1) | | | | |
| nestin (NES) | NM_006617 | −4.2 | −4.1 | −1.4 |
| fibroblast growth factor 5 (FGF5) | NM_004464 | −33 | −10 | −2 |

### REFERENCES

Gage F H: Mammalian neural stem cells, Science Feb. 25, 2000, 287(5457):1433-8

Magavi S S, Leavitt B R, Macklis J D: Induction of neurogenesis in the neocortex of adult mice, Nature. Jun. 22, 2000 405(6789):951-5

Rakic S, Zecevic N: Programmed cell death in the developing human telencephalon, Eur J Neurosci August 2000, 12(8):2721-34

Temple S, Alvarez-Buylla A: Stem cells in the adult mammalian central nervous system, Curr Opin Neurobiol February 1999, 9(1):135-41

Bain G, Kitchens D, Yao M, Huettner J E, Gottlieb D I: Embryonic stem cells express neuronal properties in vitro, Dev Biol. April 1995, 168(2):342-57

Brustle 0, Jones K N, Learish R D, Karram K, Choudhary K, Wiestler O D, Duncan I D, McKay R D: Embryonic stem cell-derived glial precursors: a source of myelinating transplants, Science Jul. 30, 1999, 285(5428):754- 6

Lindvall O, Brundin P, Widner H, Rehncrona S, Gustavii B, Frackowiak R, Leenders K L, Sawle G, Rothwell J C, Marsden C D, et al.: Grafts of fetal dopamine neurons survive and improve motor function in Parkinson's disease, Science Feb. 2, 1990, 247(4942):574-7

McKay R: Stem cells in the central nervous system, Science Apr. 4, 1997, 276(5309):66-71

Freed C R, Greene P E, Breeze R E, Tsai W Y, DuMouchel W, Kao R, Dillon S, Winfield H, Culver S, Trojanowski J Q, Eidelberg D, Fahn S: Transplantation of embryonic dopamine neurons for severe Parkinson's disease, N Engl J Med Mar, 8, 2001, 344(10):710-9

Brazelton T R, Rossi F M, Keshet G I, Blau H M: From marrow to brain: expression of neuronal phenotypes in adult mice, Science Dec. 1, 2000, 290(5497):1775-9

Mezey E, Chandross K J, Harta G, Maki R A, McKercher S R: Turning blood into brain: cells bearing neuronal antigens generated in vivo from bone marrow, Science Dec. 1, 2000, 290(5497):1779-82

Sanchez-Ramos J R: Neural cells derived from adult bone marrow and umbilical cord blood, J Neurosci Res Sep. 15, 2002, 69(6):880-93

Jiang Y, Jahagirdar B N, Reinhardt R L, Schwartz R E, Keene C D, Ortiz-Gonzalez X R, Reyes M, Lenvik T, Lund T, Blackstad M, Du J, Aldrich S, Lisberg A, Low W C, Largaespada D A, Verfaillie C M: Pluripotency of mesenchymal stem cells derived from adult marrow, Nature Jul. 4, 2002, 418(6893):41-9; Epub Jun. 20, 2002

Svendsen C N, Caldwell M A, Shen J, ter Borg M G, Rosser A E, Tyers P, Karmiol S, Dunnett S B: Long-term survival of human central nervous system progenitor cells transplanted into a rat model of Parkinson's disease, Exp Neurol November 1997, 148(1):135-46

Vescovi A L, Parati E A, Gritti A, Poulin P, Ferrario M, Wanke E, Frolichsthal-Schoeller P, Cova L, Arcellana-Panlilio M, Colombo A, Galli R: Isolation and cloning of multipotential stem cells from the embryonic human CNS and establishment of transplantable human neural stem cell lines by epigenetic stimulation, Exp Neurol March 1999, 156(1):71-83

Studer L, Tabar V, McKay R D: Transplantation of expanded mesencephalic precursors leads to recovery in parkinsonian rats, Nat Neurosci August 1998, 1 (4):290-5

Wu P, Tarasenko Y I, Gu Y, Huang L Y, Coggeshall R E, Yu Y: Region-specific generation of cholinergic neurons from fetal human neural stem cells grafted in adult rat, Nat Neurosci December 2002, 5(12):1271-8

Daadi M M, Weiss S: Generation of tyrosine hydroxylase-producing neurons from precursors of the embryonic and adult forebrain, J Neurosci Jun, 1, 1999, 19(11):4484-97

Vicario-Abejon C, Collin C, Tsoulfas P, McKay R D: Hippocampal stem cells differentiate into excitatory and inhibitory neurons, Eur J Neurosci February 2000, 12(2):677-88

Hofstetter C P, Schwarz E J, Hess D, Widenfalk J, El Manira A, Prockop D J, Olson L: Marrow stromal cells form guiding strands in the injured spinal cord and promote recovery, Proc Natl Acad Sci USA Feb. 19, 2002, 99(4):2199-204

Prockop D J, Azizi S A, Colter D, Digirolamo C, Kopen G, Phinney D G: Potential use of stem cells from bone marrow to repair the extracellular matrix and the central nervous system, Biochem Soc Trans 2000, 28(4):341-5

Toma J G, Akhavan M, Fernandes K J, Barnabe-Heider F, Sadikot A, Kaplan D R, Miller FD: Isolation of multipotent adult stem cells from the dermis of mammalian skin, Nat Cell Biol September 2001, 3(9):778-84

Kruger G M, Mosher J T, Bixby S, Joseph N, Iwashita T, Morrison S J: Neural crest stem cells persist in the adult gut but undergo changes in self-renewal, neuronal subtype potential, and factor responsiveness, Neuron Aug. 15, 2002, 35(4):657-69

Bixby S, Kruger G M, Mosher J T, Joseph N M, Morrison S J: Cell-intrinsic differences between stem cells from different regions of the peripheral nervous system regulate the generation of neural diversity, Neuron Aug, 15, 2002, 35(4):643-56

Miura M, Gronthos S, Zhao M, Lu B, Fisher L W, Robey P G, Shi S. SHED: Stem cells from human exfoliated deciduous teeth, Proc Natl Acad Sci USA May 12, 2003, 100(10):5807-12; Epub Apr. 25, 2003

Gronthos S, Brahim J, Li W, Fisher L W, Cherma n N, Boyde A, DenBesten P, Robey P G, Shi S: Stem cell properties of human dental pulp stem cells, J Dent Res August 2002, 81(8):531-5

Schierholz J, Brenner N, Zeilhofer F, Hoffmann K. H, Morsczeck C: Pluripotent embryonic-like stem cells derived from teeth and uses thereof, 2003 PCT: WO 02/066840

Bjorklund L M, Sanchez-Pernaute R, Chung S, Andersson T, Chen I Y, McNaught K S, Brownell A L, Jenkins B G, Wahlestedt C, Kim K S, Isacson O: Embryonic stem cells develop into functional dopaminergic neurons after transplantation in a Parkinson rat model, Proc Natl Acad Sci USA Feb. 19, 2002, 99(4):2344-9; Epub Jan. 8, 2002

1. A method for generating neural stem cells in vitro, comprising: isolating dental progenitor cells from soft tissue of tooth or wisdom tooth, cultivating said dental progenitor cells until they form primary spheres, dissociating said primary spheres into single cells, cultivating said single cells until they form spheroids, and separating the spheroid-forming cells to obtain neural stem cells.

2. The method of claim 1, wherein said primary spheres and said single cells, respectively, are cultured under sphere-forming conditions.

3. The method of claim 2, wherein said sphere-forming conditions are established by a medium comprising both bFGF and EGF.

4. The method of claim 3, wherein said medium further comprises B27 (1:50) or ITS+Remix (1:50).

5. The method of claim 3, wherein said medium further comprises neurobasal medium or DMEM High Glucose.

6. The method of claim 1, wherein said neural stem cells are differentiated to neural cells by incubation in differentiation-medium, preferably on coated flasks or cover slips which are preferably coated with fibronectin or poly-D-lysine and laminin.

7. The method of claim 1, wherein said dental progenitor cells are isolated from ectomesenchymal soft tissue of tooth or wisdom tooth.

8. A neural stem cell generated by the method according to claim 1.

9. A differentiated neural cell produced by the method according to claim 6.

10. A cell culture or structure comprising at least one neural stem cell according to claim 8.

11. A pharmaceutical composition comprising at least one neural stem cell according to claim 8.

12. (canceled)

13. (canceled)

14. (canceled)

15. A cell culture or structure comprising at least one differentiated neural cell according to claim 9.

16. A pharmaceutical composition comprising at least one -differentiated neural cell according to claim 9.

17. A method for treating a neurodegenerative disease comprising: administering to a subject in need thereof a neural stem cell according to claim 8 in a neurodegenerative disease treating effective amount.

18. The method of claim 17, wherein the neurodegenerative disease is selected from the group consisting of Alzheimer's disease, Parkinson's disease, prion diseases, Creutzfeldt-Jakob, Huntington's disease, multiple sclerosis, frontotemporal dementia (Pick's Disease) and amyotrophic lateral sclerosis (ALS or Lou Gehrig's Disease).
